(19) United States
(12) Patent Application Publication
(10) Pub. No.: US 2010/0129475 A1
(43) Pub. Date: May 27, 2010

(54) ESSENTIAL OIL OF KUNZEA AMBIGUA AND METHODS OF USE

(76) Inventor: John James David Hood, Waterhouse Tasmania (AU)

Correspondence Address: EDWIN D. SCHINDLER, FIVE HIRSCH AVENUE, P.O. BOX 966, CORAM, NY, 11727-0966, US

(21) Appl. No.: 12/597,372

(22) PCT Filed: Apr. 22, 2008
(86) PCT No.: PCT/AU2008/000560

§ 371 Date: Oct. 23, 2009

(30) Foreign Application Priority Data
Apr. 23, 2007 (AU) 2007902118

(51) Int. Cl. A61K 36/00

(52) U.S. Cl. 424/725

(57) ABSTRACT

A method for treating disease includes the steps of obtaining the essential oil from the Kunzea ambigua plant and administering the essential oil obtained to a human to take internally. The essential oil of the Kunzea ambigua plant has been discovered to act positively against parasitic infestation of humans and animals, and also has been found to have high kill rates against various viral and bacterial infections.

This invention related to an improved medication.

Australian Patent 721156 entitled ESSENTIAL OIL AND METHODS OF USE describes an essential oil from the kunzea ambigua and its topical use for the treatment of various conditions.

Further experimentation has shown unexpected results in the use of the oil in internal applications and the object of the present invention is to develop such use.

The invention includes the use of the essential oil from the kunzea ambigua in internal applications for the treatment of medical conditions.

Specifically it has been found that the oil acts positively against parasitic infestation of humans and animals.

The oil also has high kill rates against various viruses and germs.

Good results have been achieved by inhalation of the oil.

In order that the invention may be more readily understood, there shall be described applications of the invention.

A first patient was suffering substantial weight loss over a relatively short period (from 95 Kg to 88 Kg)

The patient was originally suspected of having a thyroid malfunction, but tests did not show more than a minor irregularity and a second diagnoses was lung cancer (although this was not indicated by X-ray).

A second opinion was sought and the medical practitioner did certain tests and weighed the patient and asked him to return for the test results after a short period.

During this period, the patient self medicated and settled on taking two doses a day of seven to eight drops of the kunzea ambigua oil in 16 mL of water, gargled and then swallowed.

The results were dramatic, the patient gained weight at a rate of some 0.5 Kg per day.

It is the patient's belief that he was, in fact, infested with parasites and the kunzea ambigua oil killed the infestation and enabled his digestive system to operate normally. The treatment was discontinued after approximately one week on the assumption that the weight gain indicated that the cause of the loss had been removed. After several years the problem has not reoccurred.

It is believed that the oil acted as a highly effective anthelmintic.

A patient who has cancer of the jaw had great success in assisting lesions caused by the cancer both externally, by using kunzea ambigua externally, together with a source of limonene and internally by applying the oil, with approximately 20 drops of oil together with a source of limonene to the lesions and swallowing the material. There have been great improvements in the lesions both in the mouth and those on the face.

A middle aged man with irritable bowel syndrome self medicated with 4 drops each of kunzea ambigua and Fragonia (agonis fragrans) oils three times a day with food. Within a week, the symptoms of the disease disappeared.

A person suffering with haemorrhoids inserted four to five drops of kunzea ambigua into his rectum twice in the first day and then daily after bowel movement and by the fifth day the symptoms were substantially relieved.

A 65 year old man had severe gout in the fingers and wrist. External application in these areas gives good results but is slow as the passage of oil through the skin in these areas is slow. He took 5-6 drops of kunzea ambigua in water or fruit juice each morning and in 11 days the swelling had regressed and the pain was substantially relieved. The client in this case also took conventional pain killers during the early part of the treatment but after five days could be stopped.

The oil has been laboratory tested against viruses and it has shown a very high kill rate. It is believed that the oil is efficacious against many diseases caused by internal germs and viruses.

Several practitioners have reported that they have had patients with cold symptoms and they have found that if these patients sniff kunzea ambigua oil during the early stages, the resulting colds have been much less severe than would have been expected and may not develop into substantial colds.

The oil can also be used as a general tonic. A few drops a day in water or fruit juice and lead to feeling of well being and can act as a stress relieving agent.

The oil can also be used to minimise jet lag.

The oil can also be successfully used against parasite attacks in animals.

In Europe, and specifically in France, essential oils are used by mainstream doctors, are prescribed by them for treating certain diseases, the prescriptions are made up by pharmacists, are used internally in many applications and are used at higher rates than have generally been the case in Britain, Australia and the United States of America.

Trials of the use of kunzea oil have been carried out in France by Dr Daniel Penoel, a leading practitioner in this field. These trials have been clinically based and have been done without external funding.

Dr Penoil states
‘Using kunzea ambigua in diluted form and in external applications can only reveal some possibilities. Obviously using higher concentrations and using it internally opens a completely new area of applications. . . . What was done with kunzea ambigua in this medical context confirmed that it has a very strong power.’

As a result of these trials, Dr Penoel has reported:

Kunzea ambigua taken internally has shown a very strong action on a certain number of viral conditions
1. kunzea ambigua has shown a strong effect on the cleansing of arteries that were partially clogged by atheromatosis plaques
2. kunzea ambigua has shown a powerful action on degenerative conditions involving certain forms of cancers (veterinary medicine).
3. kunzea ambigua has shown a very strong action on inflammatory processes, including inflammation linked with auto-immune diseases, in particular Crohn's disease.
4. kunzea ambigua has shown a very strong action, for some receptive patients, on the psyche, to give strength and resilience.
5. kunzea ambigua has shown a very strong action on consequences of chronic viral infections, like EBV (glandular fever), with results that, no pharmaceutical synthetic molecule can achieve.
6. kunzea ambigua has shown a positive action on the liver function

Dr Penoil has also carried out trials on animals and has reported:
“In veterinary medicine, it is easy to understand that many treatments can be given that would not be possible in human medicine. Several cases of dogs that should ‘normally’ have died are still living and kunzea ambigua played a major role in rescuing them. Other animals have been treated with excellent results that were found amazing by the veterinary surgeons.
If one understands that kunzea ambigua has a multiple facet action on several aspects of physiopathological processes that underlie many diseases, it is clear that the list of diseases in which it may exert a positive role is extremely vast.”

Whilst in this specification there has been described indications of the effectiveness of the oil of kunzea ambigua, it is expected that the oil can be effective against other medical conditions and such applications are deemed to be within the scope of the invention.

1-7. (canceled)

8. A method for treating disease in a human or other animal, comprising the steps of:
obtaining an essential oil of Kunzea ambigua; and,
internally administering the essential oil of Kunzea ambigua obtained in said obtaining step to a human or other animal to alleviate an infection.

9. The method for treating disease in a human or other animal according to claim 8, wherein said step of internally administering the essential oil of Kunzea ambigua to a human or other animal is via inhalation.
